(19) United States
(12) Patent Application Publication
(10) Pub. No.: US 2025/0125003 A1
(43) Pub. Date: Apr. 17, 2025

(54) GRAPH CALCULATION METHOD OF RNA SIMILARITY ANALYSIS, APPARATUS, DEVICE, AND MEDIUM

(71) Applicant: ZHEJIANG LAB, Hangzhou (CN)

(72) Inventors: Zenghui XU, Hangzhou (CN); Jin TANG, Hangzhou (CN); Yu ZHANG, Hangzhou (CN); Gaoxiang CHEN, Hangzhou (CN); Ting YU, Hangzhou (CN); Jin ZHAO, Hangzhou (CN); Ji ZHANG, Hangzhou (CN)

(21) Appl. No.: 18/608,945

(22) Filed: Mar. 19, 2024

Related U.S. Application Data
(63) Continuation of application No. PCT/CN2023/135682, filed on Nov. 30, 2023.

(30) Foreign Application Priority Data
Oct. 16, 2023 (CN) 202311335418.3

(51) Int. Cl. G16B 15/00

(52) U.S. Cl. CPC G16B 15/00

(57) ABSTRACT

A graph calculation method of RNA similarity analysis, an apparatus, a device, and a medium are provided. The method includes: converting sequence data of a looked-up RNA into a looked-up RNA structure graph; obtain a first similarity between the looked-up RNA structure graph and a target RNA structure graph; obtaining a second similarity based on the number of base constituent structures in the looked-up RNA structure graph and the number of base constituent structures in the target RNA structure graph; reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity; and obtaining a final similarity between the looked-up RNA and the target RNA based on the first similarity, the second similarity, and the third similarity.

### CROSS-REFERENCE TO RELATED APPLICATIONS

This application is a continuation of international patent application No. PCT/CN2023/135682, filed on Nov. 30, 2023, which claims priority to Chinese patent applications No. 202311335418.3, filed on Oct. 16, 2023, titled “GRAPH CALCULATION METHOD OF RNA SIMILARITY ANALYSIS, APPARATUS, DEVICE, AND MEDIUM”. The contents of the above applications are hereby incorporated by reference.

### TECHNICAL FIELD

The present disclosure generally relates to the field of bioinformatics, and in particular, to a graph calculation method of RNA similarity analysis, an apparatus, a device, and a medium.

### BACKGROUND

A Ribonucleic Acid (RNA) is a carrier of genetic information present in biological cells, some viruses, and viroids. RNA is a chain molecule condensed from ribonucleotides by phosphodiester bonds. A ribonucleotide molecule includes a phosphoric acid, a ribose, and a base. RNA mainly has four bases, that are, A (adenine), G (guanine), C (cytosine), and U (uracil).

RNA plays an important role in various biological activities. Exploration of composition and structure of RNA is one of main research directions of current biologists. RNA molecules have a plurality of stem-loop structures formed by complementary base pairs, which are secondary structures of RNA. The secondary structures can be subdivided into substructures such as 3′ fragments, 5′ fragments, hairpin loops, stems, inner loops, and multilink loop fragments.

Based on a law that there is a high probability of functional similarity in a case of RNA structural similarity, biologists mainly analyze to find similarity of secondary structures of RNAs, so as to discover other RNAs with similar functions to a target RNA, which can provide new discoveries for RNA function discovery, virus therapy, etc. The other kinds of RNAs with different similarities may be discovered by comparing the similarity of secondary structures of RNAs, so as to classify RNAs and discover new RNAs.

At present, for judgment of the similarity of secondary structures of RNAs, biologists mainly judge stem-loop structures by naked eyes and subjectively assess whether they are similar, which is subjective and inefficient. The similarity may also be judged by tree structure, wavelet analysis or other biological algorithms, but the similarity between the RNA being searched for and the target RNA cannot be calculated conveniently, intelligently, efficiently, quickly, and intuitively.

### SUMMARY

According to various embodiments of the present disclosure, a graph calculation method of RNA similarity analysis, an apparatus, a device, and a medium are provided.

In a first aspect, a graph calculation method of RNA similarity analysis is provided in an embodiment of the present disclosure, including:
converting sequence data of a looked-up RNA into a looked-up RNA structure graph;
analyzing similarity between the looked-up RNA structure graph and a target RNA structure graph to obtain a first similarity;
determining the number of base constituent structures in the looked-up RNA structure graph and obtaining a second similarity based on the number of base constituent structures in the looked-up RNA structure graph and the number of base constituent structures in the target RNA structure graph;
reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity; and
obtaining a final similarity between the looked-up RNA and the target RNA based on the first similarity, the second similarity, and the third similarity.

In an embodiment, analyzing similarity between the looked-up RNA structure graph and the target RNA structure graph to obtain the first similarity further includes:
decomposing the looked-up RNA structure graph into a plurality of looked-up RNA subgraphs by a graph kernel decomposition method, and decomposing the target RNA structure graph into a plurality of target RNA subgraphs; and
obtaining the first similarity based on the plurality of looked-up RNA subgraphs and the plurality of target RNA subgraphs.

In an embodiment, obtaining the first similarity based on the plurality of looked-up RNA subgraphs and the plurality of target RNA subgraphs further includes:
coding the plurality of looked-up RNA subgraphs to obtain a first coding sequence, and coding the plurality of target RNA subgraphs to obtain a second coding sequence; and
calculating the first similarity based on the first coding sequence and the second coding sequence.

In an embodiment, determining the number of base constituent structures in the looked-up RNA structure graph and obtaining the second similarity based on the number of base constituent structures in the looked-up RNA structure graph and the number of base constituent structures in the target RNA structure graph further includes:
determining the number of the corresponding base constituent structures in the looked-up RNA structure graph, and determining the number of the corresponding base constituent structures in the target RNA structure graph;
forming a first structure vector from the number of the corresponding base constituent structures in the looked-up RNA structure graph, and forming a second structure vector from the number of the corresponding base constituent structures in the target RNA structure graph; and
obtaining the second similarity by a Euclidean distance calculation method based on the first structure vector and the second structure vector.

In an embodiment, the number of the corresponding base constituent structures in the looked-up RNA structure graph and the number of the corresponding base constituent structures in the target RNA structure graph are determined by a graph matching algorithm.

In an embodiment, a calculation formula of obtaining the final similarity between the looked-up RNA and the target RNA based on the first similarity, the second similarity, and the third similarity is:

$$score = α * {score}_{1} + β * {score}_{2} + γ * {score}_{3} ,$$

α, β, and γ represent constraint parameters, α, β, and γ are in a range of 0 to 1, α, β, and γ satisfy a formula: α+β+γ=1, score₁ represents the first similarity, score₂ represents the second similarity, score₃ represents the third similarity, and score represents the final similarity.

In an embodiment, reconstructing the looked-up RNA structure graph based on the base constituent structures in the looked-up RNA structure graph to generate the looked-up RNA higher-order graph further includes:
taking the base constituent structures in the looked-up RNA structure graph as a node, respectively;
taking lengths of the base constituent structures as an attribute of a corresponding node, respectively; and
connecting edges based on topological relationships between the base constituent structures to form the looked-up RNA higher-order graph.

In a second aspect, a graph calculation apparatus of RNA similarity analysis is provided in an embodiment of the present disclosure, including a conversion module, a first obtaining module, a second obtaining module, a third obtaining module, and an acquiring module. The conversion module is configured for converting sequence data of a looked-up RNA into a looked-up RNA structure graph; the first obtaining module is configured for analyzing similarity between the looked-up RNA structure graph and a target RNA structure graph to obtain a first similarity; the second obtaining module is configured for determining the number of base constituent structures in the looked-up RNA structure graph and obtaining a second similarity based on the number of base constituent structures in the looked-up RNA structure graph and the number of base constituent structures in the target RNA structure graph; the third obtaining module is configured for reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity; and the acquiring module is configured for obtaining a final similarity between the looked-up RNA and the target RNA based on the first similarity, the second similarity, and the third similarity.

In a third aspect, an electronic device is provided in an embodiment of the present disclosure, including a cache module, a control module, and a plurality of computing modules. The cache module is configured for storing target RNA data, and the target RNA data includes a target RNA structure graph, a second structure vector, and a target RNA high-order graph; the control module is configured for distributing sequence data of a plurality of looked-up RNAs to the plurality of computing modules; and the plurality of computing modules are configured for computationally executing the graph calculation method of RNA similarity analysis in the first aspect based on the target RNA data and the sequence data of the plurality of looked-up RNAs to obtain similarities between the plurality of looked-up RNAs and the target RNA.

In a fourth aspect, a computer-readable storage medium is provided in an embodiment of the present disclosure. The computer-readable storage medium stores a computer program, and the computer program is executed by a processor to implement the graph calculation method of RNA similarity analysis in the first aspect.

Details of one or more embodiments of the present disclosure are set forth in the following accompanying drawings and description. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present application or the related technology, the accompanying drawings to be used in the description of the embodiments or the related technology will be briefly introduced below, and it will be obvious that the accompanying drawings in the following description are only some of the embodiments of the present application, and that, for one skilled in the art, other accompanying drawings can be obtained based on these accompanying drawings without putting in creative labor.

FIG. 1 is a schematic diagram of an application environment of a graph calculation method of RNA similarity analysis in an embodiment.

FIG. 2 is a flowchart of a graph calculation method of RNA similarity analysis in an embodiment.

FIG. 3 is sequence data of a looked-up RNA in an embodiment.

FIG. 4 is a looked-up RNA structure graph in an embodiment.

FIG. 5 is a schematic diagram of base constituent structures of a looked-up RNA structure graph in an embodiment.

FIG. 6 is a flowchart of specifical processes for performing a step 202 in an embodiment.

FIG. 7 is a flowchart of specifical processes for performing a step 302 in an embodiment.

FIG. 8 is a flowchart of specifical processes for performing a step 203 in an embodiment.

FIG. 9 is a flowchart of generating a looked-up RNA higher-order graph in an embodiment.

FIG. 10 is a looked-up RNA high-order graph in an embodiment.

FIG. 11 is a block diagram of a structure of a graph calculation apparatus of RNA similarity analysis in an embodiment.

FIG. 12 is a schematic diagram of an electronic device in an embodiment.

FIG. 13 is a flowchart of a computation process of a computing module in an embodiment.

### DETAILED DESCRIPTION OF THE EMBODIMENT

The technical solutions in the embodiments of the present disclosure will be described clearly and completely in the following in conjunction with the accompanying drawings in the embodiments of the present disclosure, and it is obvious that the described embodiments are only a part of the embodiments of the present disclosure, but not all of the embodiments. Based on the embodiments in the present disclosure, all other embodiments obtained by one skilled in the art without making creative labor fall within the scope of protection of the present disclosure.

It will be apparent that the accompanying drawings in the following description are only some examples or embodiments of the present disclosure, and that the present disclosure can be applied to other similar scenarios in accordance with these drawings, without creative effort, to one skilled in the art. In addition, it is also understood that although the efforts made in a development process may be complex and lengthy, some changes in design, manufacture, or production based on technical contents disclosed in the present disclosure are just conventional technical means for one skilled in the art related to the contents disclosed in the present disclosure, and should not be construed as inadequate disclosure of the contents disclosed in the present disclosure.

The reference to “embodiment” in the present disclosure means that a particular feature, structure, or characteristic described in connection with an embodiment may be included in at least one embodiment of the present disclosure. The presence of the phrase at various points in the specification does not necessarily refer to the same embodiment or to a separate or alternative embodiment that is mutually exclusive of other embodiments. It is understood by one skilled in the art, both explicitly and implicitly, that the embodiments described in the present disclosure may be combined with other embodiments without conflict.

Unless defined otherwise, technical terms or scientific terms involved in the present disclosure have the same meanings as would generally understood by one skilled in the technical field of the present disclosure. In the present disclosure, “a”, “an”, “one”, “the”, and other similar words do not indicate a quantitative limitation, which may be singular or plural. The terms such as “comprise”, “include”, “have”, and any variants thereof involved in the present disclosure are intended to cover a non-exclusive inclusion. For example, processes, methods, systems, products, or devices including a series of steps or modules (units) are not limited to these steps or modules (units) listed, and may include other steps or modules (units) not listed, or may include other steps or modules (units) inherent to these processes, methods, systems, products, or devices. Words such as “join”, “connect”, “couple”, and the like involved in the present disclosure are not limited to physical or mechanical connections, and may include electrical connections, whether direct or indirect. “A plurality of” involved in the present disclosure means two or more. The term “and/or” describes an association relationship between associated objects and represents that three relationships may exist. For example, A and/or B may represent the following three cases: only A exists, both A and B exist, and only B exists. Generally, a character “/” indicates an “or” relationship between the associated objects. The terms “first”, “second”, “third”, and the like involved in the present disclosure are only intended to distinguish similar objects and do not represent specific ordering of the objects.

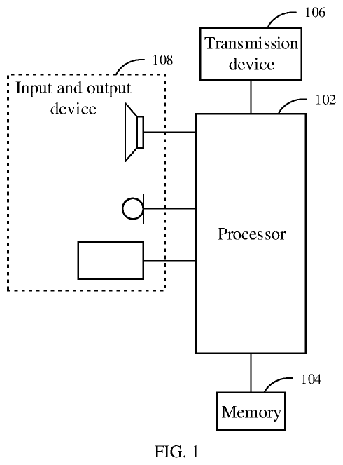

A method embodiment provided in the embodiment may be executed in a terminal, a computer, or a similar computing device. For example, the method may be performed on a terminal. FIG. 1 is a schematic diagram of an application environment of a graph calculation method of RNA similarity analysis in an embodiment. Referring to FIG. 1, the terminal may include one or more (only one is shown in FIG. 1) processors 102 and a memory 104 for storing data. The processors 102 may include, but are not limited to, processing devices such as Microcontroller Unit (MCU) or Field Programmable Gate Array (FPGA). The terminal may also include a transmission device 106 for communication functions and an input/output device 108. It may be understood by one skilled in the art that the structure shown in FIG. 1 is merely illustrative, and that it does not impose a limitation on the structure of the terminal. For example, the terminal may also include more or fewer components than shown in FIG. 1, or have a different configuration than that illustrated in FIG. 1.

The memory 104 may be configured to store a computer program, e.g., a software program and a module of an application software, such as a computer program corresponding to the graph calculation method of RNA similarity analysis in the present embodiment, and the processor 102 may perform various functional applications and data processing by running the computer program stored in the memory 104, i.e., realizes the method described above. The memory 104 may include a high-speed random memory, and may also include a non-volatile memory, such as one or more magnetic storage devices, flash memories, or other non-volatile solid-state memories. In some examples, the memory 104 may further include memories set remotely relative to the processor 102, and these remote memories may be connected to the terminal via a network. Examples of the network may include, but are not limited to, the Internet, an enterprise intranet, a local area network, a mobile communications network, and combinations thereof.

The transmission device 106 is configured to receive or send data via a network. The network may include a wireless network provided by a communication provider of the terminal. In an example, the transmission device 106 may include a Network Interface Controller (NIC) that can be connected to other network devices via a base station and thus can be in communication with the Internet. In an example, the transmission device 106 may be a radio frequency (RF) module that is configured to be in communication with the Internet wirelessly.

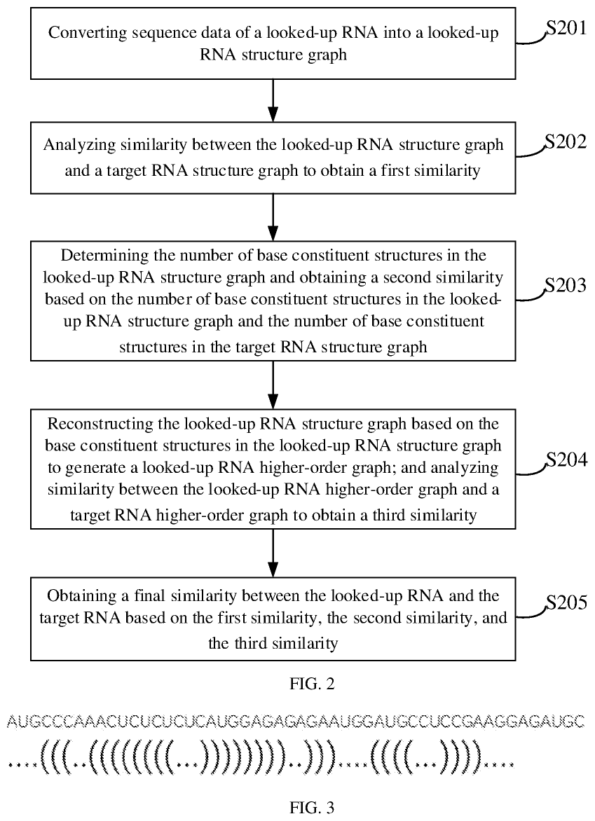

In a first aspect, a graph calculation method of RNA similarity analysis is provided in an embodiment of the present disclosure. Referring to FIG. 2, the method includes following step 201 to step 205.

Step 201 includes converting sequence data of a looked-up RNA into a looked-up RNA structure graph.

Specifically, a RNA sequence may include four bases, that are A (adenine), G (guanine), C (cytosine), and U (uracil), the RNA sequence data may include the RNA sequence and a dot bracket sequence, and the dot bracket sequence may be secondary structural information of the RNA expressed by dots and pairs of parentheses. Free bases in the RNA sequence that do not have complementary base pair may be represented by the dots “.”, and two bases that form a complementary base pair may be represented by a pair of brackets “( )”. Exemplarily, FIG. 3 is sequence data of a looked-up RNA.

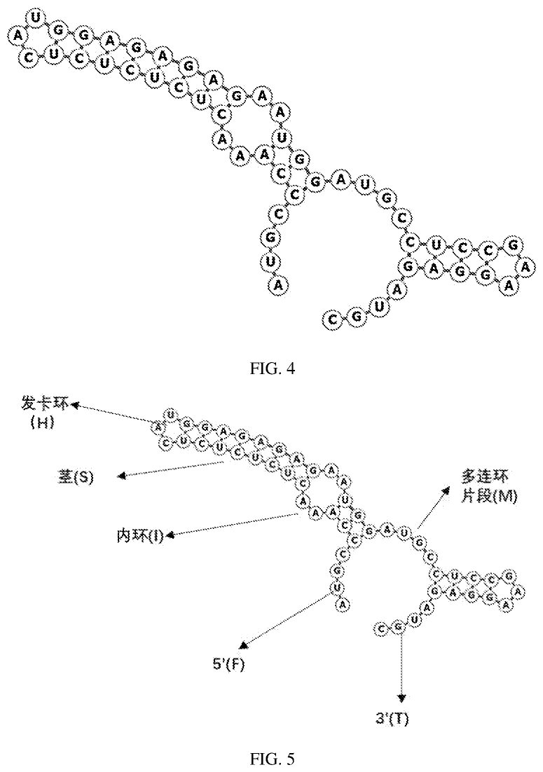

Converting sequence data of the looked-up RNA into a looked-up RNA structure graph may further include: based on the secondary structural information of the RNA included in the sequence data of the looked-up RNA, converting the sequence data of the looked-up RNA into a graph structure representation in the field of computational science. Each base in the sequence data of the looked-up RNA may be taken as a graph node, four characteristics of A, G, C, and U of the bases may be taken as attributes of the nodes, and base nodes may be connected by relationships between secondary structures. Exemplarily, FIG. 4 is a looked-up RNA structure graph based on the conversion of the sequence data of the looked-up RNA.

Step 202 includes analyzing similarity between the looked-up RNA structure graph and a target RNA structure graph to obtain a first similarity.

Specifically, the target RNA structure graph in the present embodiment may also be obtained based on the sequence data of the target RNA by the same method as the step 201. The present embodiment may adopt an analysis method of graph similarity to analyze the similarity between the looked-up RNA structure graph and the target RNA structure graph to obtain the first similarity.

Step 203 includes determining the number of base constituent structures in the looked-up RNA structure graph and obtaining a second similarity based on the number of base constituent structures in the looked-up RNA structure graph and the number of base constituent structures in the target RNA structure graph.

The base constituent structures in the RNA structure graph may include: a 3′ fragment, a 5′ fragment, a hairpin loop, a stem, an inner loop, and a multilink loop fragment. Exemplarily, FIG. 5 is a schematic diagram of base constituent structures of a looked-up RNA structure graph.

Step 204 includes reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity.

Specifically, in the present embodiment, the looked-up RNA structure graph may be reconstructed based on the base constituent structures in the looked-up RNA structure graph to generate the looked-up RNA higher-order graph. Prior to this, the target RNA structure graph may also be reconstructed based on the base constituent structures in the target RNA structure graph to generate a target RNA high-order graph. The analysis method of graph similarity may also be adopted in this step to analyze the similarity between the looked-up RNA higher-order graph and the target RNA higher-order graph to obtain the third similarity.

Step 205 includes obtaining a final similarity between the looked-up RNA and the target RNA based on the first similarity, the second similarity, and the third similarity.

At the step 201 to the step 205, the sequence data of the looked-up RNA may be converted into the looked-up RNA structure graph; the similarity between the looked-up RNA structure graph and the target RNA structure graph may be analyzed to obtain the first similarity; the number of base constituent structures in the looked-up RNA structure graph may be determined and the second similarity may be obtained based on the number of base constituent structures in the looked-up RNA structure graph and the number of base constituent structures in the target RNA structure graph; the looked-up RNA structure graph may be reconstructed based on the base constituent structures in the looked-up RNA structure graph to generate the looked-up RNA high-order graph; the similarity between the looked-up RNA high-order graph and the target RNA high-order graph may be analyzed to obtain the third similarity; and the final similarity between the looked-up RNA and the target RNA may be obtained based on the first similarity, the second similarity, and the third similarity. In the present disclosure, the secondary structures of RNA may be represented as a graph structure in the field of computational science, a graph analysis method may be applied to an analysis field of the secondary structures of RNA, and a graph data structure in the field of computational science may be used to intuitively describe the secondary structures of RNA. A variety of graph similarity methods such as an analysis method of graph similarity, a graph structure analysis, and a higher-order graph analysis may be introduced to secondary structure analysis of RNA, a variety of dimensions of information such as graph similarity analysis, RNA secondary structure composition, higher-order graph information may be comprehensively considered to calculate the similarity between RNAs, a calculation result may be more credible, and intelligent and automatic RNA similarity calculation may be realized, which greatly facilitates biologists to discover other RNAs with similar functions to the target RNA, as well as to discover other kinds of RNAs with different similarities to the target RNA, so as to classify RNAs and discover new RNAs.

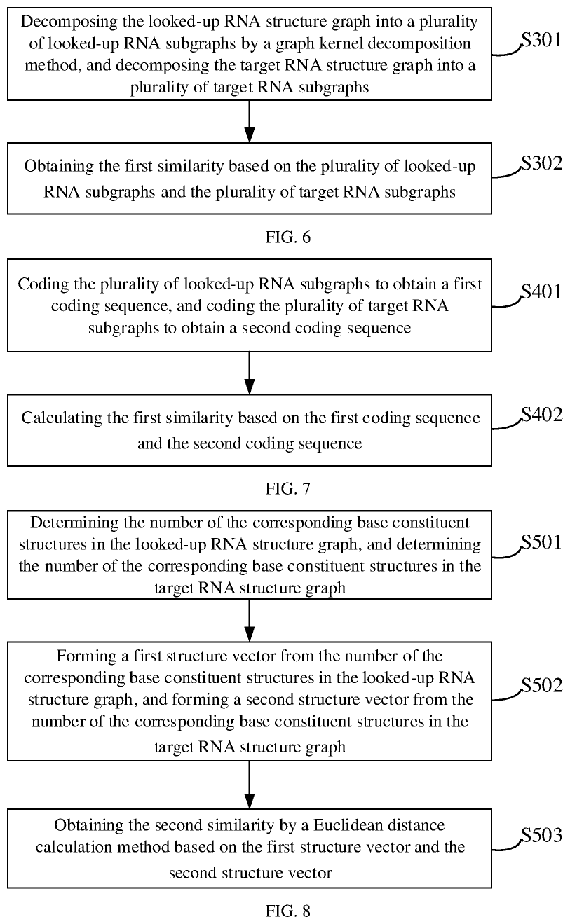

In an embodiment, referring to FIG. 6, analyzing similarity between the looked-up RNA structure graph and the target RNA structure graph to obtain the first similarity may further include following step 301 and step 302.

Step 301 may include decomposing the looked-up RNA structure graph into a plurality of looked-up RNA subgraphs by a graph kernel decomposition method, and decomposing the target RNA structure graph into a plurality of target RNA subgraphs.

Step 302 may include obtaining the first similarity based on the plurality of looked-up RNA subgraphs and the plurality of target RNA subgraphs.

In an embodiment, referring to FIG. 7, obtaining the first similarity based on the plurality of looked-up RNA subgraphs and the plurality of target RNA subgraphs may further includes following step 401 and step 402.

Step 401 may include coding the plurality of looked-up RNA subgraphs to obtain a first coding sequence, and coding the plurality of target RNA subgraphs to obtain a second coding sequence.

Step 402 may include calculating the first similarity based on the first coding sequence and the second coding sequence. A specifical calculation formula of calculating the first similarity denoted as score₁ by a jaccard method is as follows:

$${L}_{t}^{n e w} = {K}_{W L} ( {G}_{t} ) , {L}_{u}^{n e w} = {K}_{W L} ( {G}_{u} ) ;$$

$${score}_{1} = jaccard ( {L}_{t}^{n e w} , {L}_{u}^{n e w} ) = \frac{\vert {L}_{t}^{n e w} ⋂ {L}_{u}^{n e w} \vert}{\vert {L}_{t}^{n e w} ⋃ {L}_{u}^{n e w} \vert} ,$$

Gₜ represents the target RNA structure graph, Gᵤ represents the looked-up RNA structure graph, K_{WL} represents a WL (Weisfeiler-Lehman) kernel method, Lₜ^{new} represents the second coding sequence, and Lᵤ^{new} represents the first coding sequence.

The graph kernel decomposition method in the present embodiment may employ the WL (Weisfeiler-Lehman) kernel method to decompose the looked-up RNA structure graph into the plurality of looked-up RNA subgraphs, and to decompose the target RNA structure graph into the plurality of target RNA sub-graphs. Taking the looked-up RNA structure graph as an example, specific decomposition may be to decompose each node in the structure graph and the nodes adjacent to that node into a subgraph. When the looked-up RNA structure graph has 50 bases, the looked-up RNA structure graph may be decomposed into 50 subgraphs, and each subgraph may include a node and nodes adjacent to the node. The coding of each node (e.g., A: #1000, G: #1001, C: #1002, U: #1003) may be performed according to attributes of nodes (A (adenine), G (guanine), C (cytosine), and U (uracil)), the coding of each subgraph may be obtained based on the coding of each node, a first coding sequence may be obtained according to the coding of looked-up RNA subgraphs, and a second coding sequence may be obtained according to the coding of target RNA subgraphs.

In an embodiment, referring to FIG. 8, determining the number of base constituent structures in the looked-up RNA structure graph and obtaining the second similarity based on the number of base constituent structures in the looked-up RNA structure graph and the number of base constituent structures in the target RNA structure graph may further include following step 501 to step 503.

Step 501 may include determining the number of the corresponding base constituent structures in the looked-up RNA structure graph, and determining the number of the corresponding base constituent structures in the target RNA structure graph.

Step 502 may include forming a first structure vector from the number of the corresponding base constituent structures in the looked-up RNA structure graph, and forming a second structure vector from the number of the corresponding base constituent structures in the target RNA structure graph.

Step 503 may include obtaining the second similarity by a Euclidean distance calculation method based on the first structure vector and the second structure vector.

Specifically, a specific formula for calculating the second similarity may be as follows:

Find(Gₜ)=[Tₜ,Fₜ,Hₜ,Sₜ,Iₜ,Mₜ];

Find(Gᵤ)=[Tᵤ,Fᵤ,Hᵤ,Sᵤ,Iᵤ,Mᵤ];

score₂=E_distan([Tₜ,Fₜ,Hₜ,Sₜ,Iₜ,Mₜ],[Tᵤ,Fᵤ,Hᵤ,Sᵤ,Iᵤ,Mᵤ]),

Find represents a graph structure finding method, Gₜ represents the target RNA structure graph, Gᵤ represents the looked-up RNA structure graph, Tₜ, Fₜ, Hₜ, Sₜ, Iₜ, and Mₜ represent the number of 3′ fragments, 5′ fragments, hairpin loops, stems, inner loops, and multilink loop fragments contained in the target RNA structure graph obtained by the graph structure finding method, respectively; Tᵤ, Fᵤ, Hᵤ, Sᵤ, Iᵤ, Mᵤ represent the number of 3′ fragments, 5′ fragments, hairpin loops, stems, inner loops, and multilink loop fragments contained in the looked-up RNA structure graph obtained by the graph structure finding method, respectively; [Tᵤ, Fᵤ, Hᵤ, Sᵤ, Iᵤ, Mᵤ] represents the first structure vector including the number of base constituent structures in the looked-up RNA structure graph; [Tₜ, Fₜ, Hₜ, Sₜ, Iₜ, Mₜ] represents the second structure vector including the number of base constituent structures in the target RNA structure graph, E_distan represents the Euclidean distance, and score₂ represents a structural similarity score of two graphs, i.e., the second similarity.

In an embodiment, the graph structure finding method may use a Graph Matching algorithm in the field of graph computation to realize a search for the looked-up RNA structure graph and the target RNA structure graph. In the present disclosure, the number of the corresponding base constituent structures in the looked-up RNA structure graph and the number of the corresponding base constituent structures in the target RNA structure graph may be determined by employing the Graph Matching algorithm.

In an embodiment, a calculation formula of obtaining the final similarity between the looked-up RNA and the target RNA based on the first similarity, the second similarity, and the third similarity may be:

$$score = α * {score}_{1} + β * {score}_{2} + γ * {score}_{3} ,$$

α, β, and γ represent constraint parameters, α, β, and γ are in a range of 0 to 1, α, β, and γ satisfy a formula: α+β+γ=1, score₁ represents the first similarity, score₂ represents the second similarity, score₃ represents the third similarity, and score represents the final similarity.

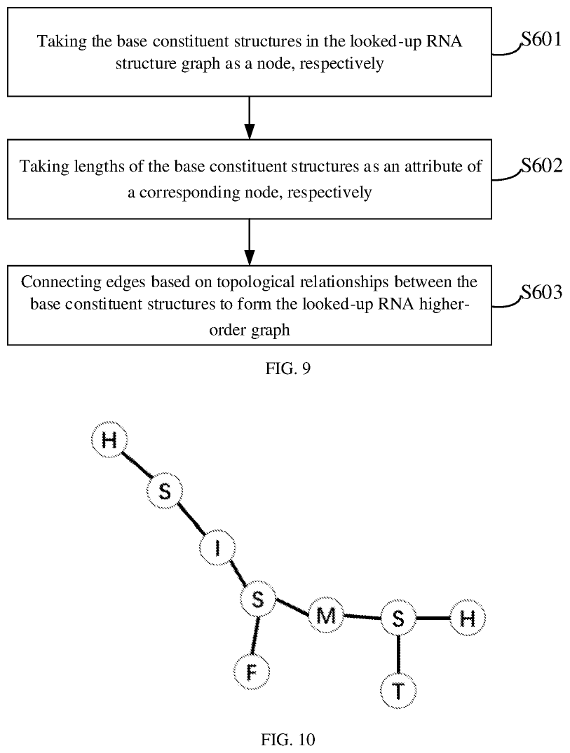

In an embodiment, referring to FIG. 9, reconstructing the looked-up RNA structure graph based on the base constituent structures in the looked-up RNA structure graph to generate the looked-up RNA higher-order graph may further include following step 601 to step 603.

Step 601 may include taking the base constituent structures in the looked-up RNA structure graph as a node, respectively.

Step 602 may include taking lengths of the base constituent structures as an attribute of a corresponding node, respectively.

Step 603 may include connecting edges based on topological relationships between the base constituent structures to form the looked-up RNA higher-order graph.

Specifically, 3′ fragments, 5′ fragments, the hairpin loops, the stems, the inner loops, and the multilink loop fragments in the looked-up RNA structure graph may be taken as a node in the graph structure, respectively. The lengths of the base constituent structures may be taken as the number of bases composing the base constituent structures, for example, when the base constituent structure has 5 bases, the attribute of the node may be 5. Connecting edges may be performed based on the topological relationships between the base constituent structures, and the topological relationships between the base constituent structures may be known from the looked-up RNA structure graph. Prior to this, the target RNA structure graph may also be reconstructed based on the base constituent structures in the target RNA structure graph to generate the target RNA higher-order graph.

Exemplarily, FIG. 10 is the looked-up RNA higher-order graph generated based on the looked-up RNA structure graph. In the FIG. 10, H represents a hairpin loop, S represents a stem, I represents an inner loop, M represents a multilink loop fragment, F represents a 5′ fragment, and T represents a 3′ fragment.

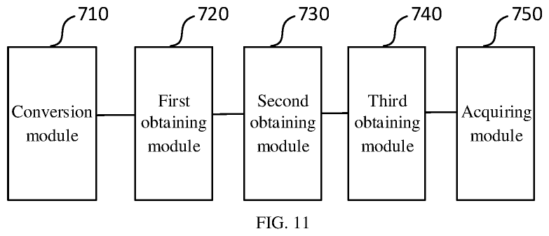

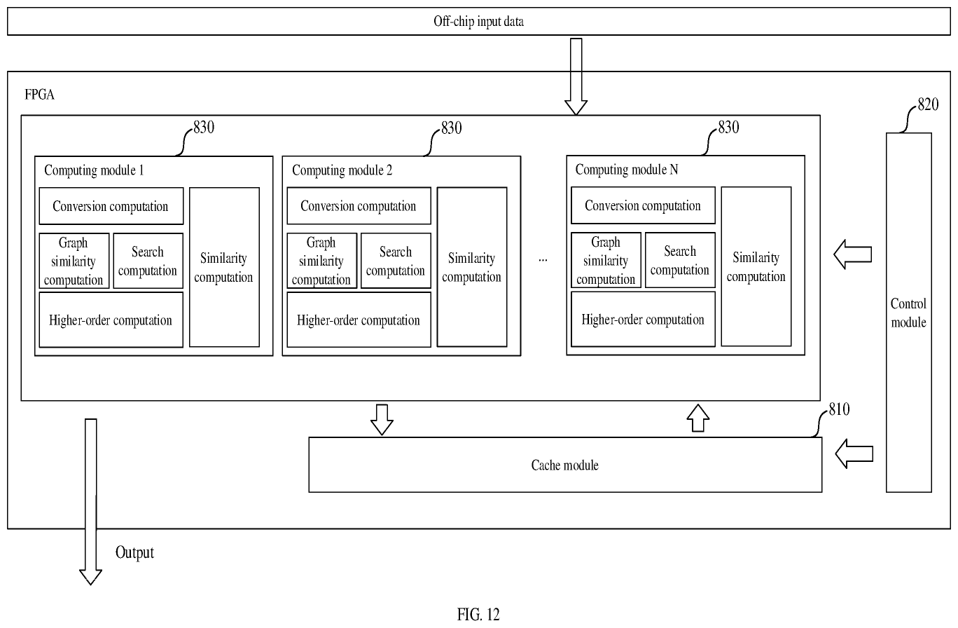

A graph calculation apparatus of RNA similarity analysis is provided in an embodiment of the present disclosure, referring to FIG. 11, the apparatus includes a conversion module 710, a first obtaining module 720, a second obtaining module 730, a third obtaining module 740, and an acquiring module 750. The conversion module 710 is configured for converting sequence data of a looked-up RNA into a looked-up RNA structure graph. The first obtaining module 720 is configured for analyzing similarity between the looked-up RNA structure graph and a target RNA structure graph to obtain a first similarity. The second obtaining module 730 is configured for determining the number of base constituent structures in the looked-up RNA structure graph and obtaining a second similarity based on the number of base constituent structures in the looked-up RNA structure graph and the number of base constituent structures in the target RNA structure graph. The third obtaining module 740 is configured for reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity. The acquiring module 750 is configured for obtaining a final similarity between the looked-up RNA and the target RNA based on the first similarity, the second similarity, and the third similarity.

In an embodiment, the first obtaining module 720 is further configured for decomposing the looked-up RNA structure graph into a plurality of looked-up RNA subgraphs by a graph kernel decomposition method, and decomposing the target RNA structure graph into a plurality of target RNA subgraphs; and obtaining the first similarity based on the plurality of looked-up RNA subgraphs and the plurality of target RNA subgraphs.

In an embodiment, the first getting module 720 is further configured for coding the plurality of looked-up RNA subgraphs to obtain a first coding sequence, and coding the plurality of target RNA subgraphs to obtain a second coding sequence; and calculating the first similarity based on the first coding sequence and the second coding sequence.

In an embodiment, the second obtaining module 730 is further configured for determining the number of the corresponding base constituent structures in the looked-up RNA structure graph, and determining the number of the corresponding base constituent structures in the target RNA structure graph; forming a first structure vector from the number of the corresponding base constituent structures in the looked-up RNA structure graph, and forming a second structure vector from the number of the corresponding base constituent structures in the target RNA structure graph; and based on the first structure vector and the second structure vector, obtaining the second similarity by a Euclidean distance calculation method.

In an embodiment, the second obtaining module 730 is further configured for determine a corresponding number of each underlying constituent structure in the structural graph of the looked-up RNA and in the structural graph of the target RNA using a graph matching algorithm.

In an embodiment, a calculation formula of obtaining the final similarity between the looked-up RNA and the target RNA based on the first similarity, the second similarity, and the third similarity may be as follows:

$$score = α * {score}_{1} + β * {score}_{2} + γ * {score}_{3} ,$$

α, β, and γ represent constraint parameters, α, β, and γ are in a range of 0 to 1, α, β, and γ satisfy a formula: α+β+γ=1, score₁, represents the first similarity, score₂ represents the second similarity, score₃ represents the third similarity, and score represents the final similarity.

In an embodiment, the third obtaining module 740 is further configured for taking the base constituent structures in the looked-up RNA structure graph as a node, respectively; taking lengths of the base constituent structures as an attribute of a corresponding node, respectively; and connecting edges based on topological relationships between the base constituent structures to form the looked-up RNA higher-order graph.

It should be noted that each of the above-described modules may be a function module or a program module, and may be implemented either by software or by hardware. For the modules realized by hardware, the above-described individual modules may be disposed in the same processor; or the above-described individual modules may also be disposed in different processors according to any combination.

In a third aspect, an electronic device based on FPGA (Field Programmable Gate Array) hardware is provided in an embodiment of the present disclosure. Referring to FIG. 12, the electronic device includes a cache module 810, a control module 820, and a plurality of computing modules 830.

The cache module 810 is configured for storing target RNA data, and the target RNA data includes a target RNA structure graph, a second structure vector, and a target RNA high-order graph.

Specifically, in the present embodiment, the cache module 810 may be an on-chip cache. The sequence data of the target RNA may be first input into the plurality of computing modules 830, and after conversion computation, graph similarity computation, search computation, and higher-order computation, a decomposition subgraph of the secondary structure of the target RNA, a second structure vector [Tₜ, Fₜ, Hₜ, Sₜ, Iₜ, Mₜ], and a decomposition subgraph of the target RNA higher-order graph may be obtained, respectively, and be stored in the on-chip cache.

The control module 820 is configured for distributing sequence data of a plurality of looked-up RNAs to the plurality of computing modules 830.

Specifically, large-scale to-be-analyzed sequence data of looked-up RNA which is inputted off-chip may be distributed to N computing modules 830 by the control module 820, and corresponding solving computations may be performed in the N computing modules 830 in a streaming manner.

The plurality of computing modules 830 are configured for computationally executing steps of the graph calculation method of RNA similarity analysis in any one of the above-mentioned embodiments based on the target RNA data and the sequence data of the plurality of looked-up RNAs, to obtain similarities between the plurality of looked-up RNAs and the target RNA.

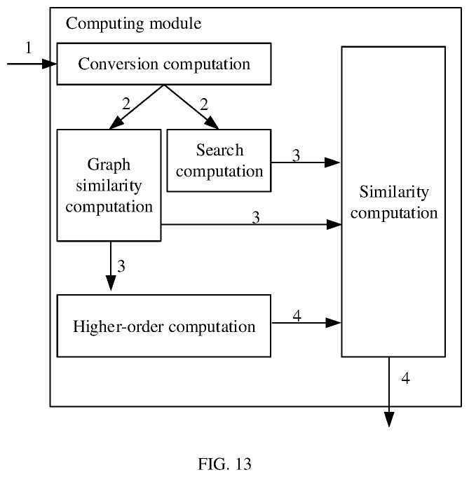

Referring to FIG. 13, a specific solution computation process of the plurality of computing modules 830 may be as follows: firstly, a conversion computation may be performed, the conversion computation is configured to convert the sequence data of the looked-up RNAs into looked-up RNA structure graphs to obtain the looked-up RNA structure graphs, then the looked-up RNA structure graphs may be simultaneously distributed to the graph similarity computation and the search computation to execute subgraph decomposition and structure vector solving in parallel, the result may be passed to a similarity computation and the higher-order computation, then the decomposition subgraph of the higher-order graph may be simultaneously solved in higher-order computation, the first similarity score₁ and the second similarity score₂ may be solved in the similarity computation, and finally the third similarity scores and the final similarity score may be calculated in the similarity computation and output. Decomposition subgraphs of secondary structures, structure vectors, and decomposition subgraphs of higher-order graphs may be solved to obtain the final similarity in the similarity calculation.

In the present embodiment, a plurality of parallel computing modules 830 may be disposed in the electronic device for streaming computation of large-scale similarities between the looked-up RNAs and the target RAN, and perform similarity analysis of large-scale RNA structures, realizing accelerated computation. The plurality of computing modules 830 in the whole computational process may use parallel and streaming computation, and make full use of the plurality of computing modules 830, with high computational efficiency, realizing accelerated computation.

In an embodiment, a computer-readable storage medium is provided. The computer-readable storage medium stores a computer program, and the computer program is executed by a processor to implement the steps of the graph calculation method of RNA similarity analysis in any one of the above embodiments.

One skilled in the art may understand that realizing all or part of the processes in the methods of the above embodiments is possible by means of a computer program to instruct the relevant hardware to accomplish the same, the computer program may be stored in a non-volatile computer-readable storage medium, and the computer program may include processes such as the processes of the embodiments of the methods described above when executed. Any reference to a memory, storage, database, or other medium used in the embodiments provided in the present disclosure may include at least one of non-volatile and volatile memories. The Non-volatile memory may include a Read Only Memory (ROM), a magnetic tape, a floppy disk, a flash memory, or an optical memory. The volatile memory may include a Random Access Memory (RAM) or an external cache memory. As an illustration and not as a limitation, the RAM may be in various forms, such as Static Random Access Memory (SRAM) or Dynamic Random Access Memory (DRAM), and the like.

The various technical features of the above-described embodiments may be combined arbitrarily, and all possible combinations of the various technical features of the above-described embodiments have not been described for the sake of conciseness of description. However, as long as there is no contradiction in the combinations of these technical features, they should be considered to be within the scope of the present specification.

The above-described embodiments express only several embodiments of the present disclosure, which are described in a more specific and detailed manner, but are not to be construed as a limitation on the scope of the present disclosure. For one skilled in the art, several deformations and improvements can be made without departing from the conception of the present disclosure, all of which fall within the scope of protection of the present disclosure. Therefore, the scope of protection of the present disclosure shall be subject to the attached claims.

What is claimed is:

1. A graph calculation method of RNA similarity analysis, comprising:
converting sequence data of a looked-up RNA into a looked-up RNA structure graph;
analyzing similarity between the looked-up RNA structure graph and a target RNA structure graph to obtain a first similarity;
determining the number of base constituent structures in the looked-up RNA structure graph and obtaining a second similarity based on the number of base constituent structures in the looked-up RNA structure graph and the number of base constituent structures in the target RNA structure graph;
reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity; and
obtaining a final similarity between the looked-up RNA and the target RNA based on the first similarity, the second similarity, and the third similarity.

2. The graph calculation method of RNA similarity analysis of claim 1, wherein analyzing similarity between the looked-up RNA structure graph and the target RNA structure graph to obtain the first similarity further comprises:
decomposing the looked-up RNA structure graph into a plurality of looked-up RNA subgraphs by a graph kernel decomposition method, and decomposing the target RNA structure graph into a plurality of target RNA subgraphs; and
obtaining the first similarity based on the plurality of looked-up RNA subgraphs and the plurality of target RNA subgraphs.

3. The graph calculation method of RNA similarity analysis of claim 2, wherein obtaining the first similarity based on the plurality of looked-up RNA subgraphs and the plurality of target RNA subgraphs further comprises:
coding the plurality of looked-up RNA subgraphs to obtain a first coding sequence, and coding the plurality of target RNA subgraphs to obtain a second coding sequence; and
calculating the first similarity based on the first coding sequence and the second coding sequence.

4. The graph calculation method of RNA similarity analysis of claim 1, wherein determining the number of base constituent structures in the looked-up RNA structure graph and obtaining the second similarity based on the number of base constituent structures in the looked-up RNA structure graph and the number of base constituent structures in the target RNA structure graph further comprises:
determining the number of the corresponding base constituent structures in the looked-up RNA structure graph, and determining the number of the corresponding base constituent structures in the target RNA structure graph;
forming a first structure vector from the number of the corresponding base constituent structures in the looked-up RNA structure graph, and forming a second structure vector from the number of the corresponding base constituent structures in the target RNA structure graph; and
obtaining the second similarity by a Euclidean distance calculation method based on the first structure vector and the second structure vector.

5. The graph calculation method of RNA similarity analysis of claim 4, wherein the number of the corresponding base constituent structures in the looked-up RNA structure graph and the number of the corresponding base constituent structures in the target RNA structure graph are determined by a graph matching algorithm.

6. The graph calculation method of RNA similarity analysis of claim 1, wherein a calculation formula of obtaining the final similarity between the looked-up RNA and the target RNA based on the first similarity, the second similarity, and the third similarity is:
$$score = α * {score}_{1} + β * {score}_{2} + γ * {score}_{3} ,$$
wherein α, β, and γ represent constraint parameters, α, β, and γ are in a range of 0 to 1, α, β, and γ satisfy a formula: α+β+γ=1, score₁ represents the first similarity, score₂ represents the second similarity, score₃ represents the third similarity, and score represents the final similarity.

7. The graph calculation method of RNA similarity analysis of claim 1, wherein reconstructing the looked-up RNA structure graph based on the base constituent structures in the looked-up RNA structure graph to generate the looked-up RNA higher-order graph further comprises:
taking the base constituent structures in the looked-up RNA structure graph as a node, respectively;
taking lengths of the base constituent structures as an attribute of a corresponding node, respectively; and
connecting edges based on topological relationships between the base constituent structures to form the looked-up RNA higher-order graph.

8. A graph calculation apparatus of RNA similarity analysis, comprising a conversion module, a first obtaining module, a second obtaining module, a third obtaining module, and an acquiring module,
wherein the conversion module is configured for converting sequence data of a looked-up RNA into a looked-up RNA structure graph;
the first obtaining module is configured for analyzing similarity between the looked-up RNA structure graph and a target RNA structure graph to obtain a first similarity;
the second obtaining module is configured for determining the number of base constituent structures in the looked-up RNA structure graph and obtaining a second similarity based on the number of base constituent structures in the looked-up RNA structure graph and the number of base constituent structures in the target RNA structure graph;
the third obtaining module is configured for reconstructing the looked-up RNA structure graph based on the base constituent structures in the looked-up RNA structure graph to generate a looked-up RNA higher-order graph; and analyzing similarity between the looked-up RNA higher-order graph and a target RNA higher-order graph to obtain a third similarity; and
the acquiring module is configured for obtaining a final similarity between the looked-up RNA and the target RNA based on the first similarity, the second similarity, and the third similarity.

9. An electronic device, comprising a cache module, a control module, and a plurality of computing modules;
wherein the cache module is configured for storing target RNA data, and the target RNA data comprises a target RNA structure graph, a second structure vector, and a target RNA high-order graph;
the control module is configured for distributing sequence data of a plurality of looked-up RNAs to the plurality of computing modules; and
the plurality of computing modules are configured for computationally executing the graph calculation method of RNA similarity analysis of claim 1 based on the target RNA data and the sequence data of the plurality of looked-up RNAs to obtain similarities between the plurality of looked-up RNAs and the target RNA.

10. A computer-readable storage medium, storing a computer program, wherein the computer program is executed by a processor to implement the graph calculation method of RNA similarity analysis of claim 1.

11. The electronic device of claim 9, wherein analyzing similarity between the looked-up RNA structure graph and the target RNA structure graph to obtain the first similarity further comprises:
decomposing the looked-up RNA structure graph into a plurality of looked-up RNA subgraphs by a graph kernel decomposition method, and decomposing the target RNA structure graph into a plurality of target RNA subgraphs; and
obtaining the first similarity based on the plurality of looked-up RNA subgraphs and the plurality of target RNA subgraphs.

12. The electronic device of claim 11, wherein obtaining the first similarity based on the plurality of looked-up RNA subgraphs and the plurality of target RNA subgraphs further comprises:
coding the plurality of looked-up RNA subgraphs to obtain a first coding sequence, and coding the plurality of target RNA subgraphs to obtain a second coding sequence; and
calculating the first similarity based on the first coding sequence and the second coding sequence.

13. The electronic device of claim 9, wherein determining the number of base constituent structures in the looked-up RNA structure graph and obtaining the second similarity based on the number of base constituent structures in the looked-up RNA structure graph and the number of base constituent structures in the target RNA structure graph further comprises:
determining the number of the corresponding base constituent structures in the looked-up RNA structure graph, and determining the number of the corresponding base constituent structures in the target RNA structure graph;
forming a first structure vector from the number of the corresponding base constituent structures in the looked-up RNA structure graph, and forming a second structure vector from the number of the corresponding base constituent structures in the target RNA structure graph; and
obtaining the second similarity by a Euclidean distance calculation method based on the first structure vector and the second structure vector.

14. The electronic device of claim 13, wherein the number of the corresponding base constituent structures in the looked-up RNA structure graph and the number of the corresponding base constituent structures in the target RNA structure graph are determined by a graph matching algorithm.

15. The electronic device of claim 9, wherein a calculation formula of obtaining the final similarity between the looked-up RNA and the target RNA based on the first similarity, the second similarity, and the third similarity is:
$$score = α * {score}_{1} + β * {score}_{2} + γ * {score}_{3} ,$$
wherein α, β, and γ represent constraint parameters, α, β, and γ are in a range of 0 to 1, α, β, and γ satisfy a formula: α+β+γ=1, score₁ represents the first similarity, score₂ represents the second similarity, score₃ represents the third similarity, and score represents the final similarity.

16. The electronic device of claim 9, wherein reconstructing the looked-up RNA structure graph based on the base constituent structures in the looked-up RNA structure graph to generate the looked-up RNA higher-order graph further comprises:
taking the base constituent structures in the looked-up RNA structure graph as a node, respectively;
taking lengths of the base constituent structures as an attribute of a corresponding node, respectively; and
connecting edges based on topological relationships between the base constituent structures to form the looked-up RNA higher-order graph.

17. The computer-readable storage medium of claim 10, wherein analyzing similarity between the looked-up RNA structure graph and the target RNA structure graph to obtain the first similarity further comprises:
decomposing the looked-up RNA structure graph into a plurality of looked-up RNA subgraphs by a graph kernel decomposition method, and decomposing the target RNA structure graph into a plurality of target RNA subgraphs; and
obtaining the first similarity based on the plurality of looked-up RNA subgraphs and the plurality of target RNA subgraphs.

18. The computer-readable storage medium of claim 17, wherein obtaining the first similarity based on the plurality of looked-up RNA subgraphs and the plurality of target RNA subgraphs further comprises:
coding the plurality of looked-up RNA subgraphs to obtain a first coding sequence, and coding the plurality of target RNA subgraphs to obtain a second coding sequence; and
calculating the first similarity based on the first coding sequence and the second coding sequence.

19. The computer-readable storage medium of claim 10, wherein determining the number of base constituent structures in the looked-up RNA structure graph and obtaining the second similarity based on the number of base constituent structures in the looked-up RNA structure graph and the number of base constituent structures in the target RNA structure graph further comprises:
determining the number of the corresponding base constituent structures in the looked-up RNA structure graph, and determining the number of the corresponding base constituent structures in the target RNA structure graph;
forming a first structure vector from the number of the corresponding base constituent structures in the looked-up RNA structure graph, and forming a second structure vector from the number of the corresponding base constituent structures in the target RNA structure graph; and
obtaining the second similarity by a Euclidean distance calculation method based on the first structure vector and the second structure vector.

20. The computer-readable storage medium of claim 19, wherein the number of the corresponding base constituent structures in the looked-up RNA structure graph and the number of the corresponding base constituent structures in the target RNA structure graph are determined by a graph matching algorithm.
